(19) United States
(12) Patent Application Publication
(10) Pub. No.: US 2022/0265833 A1
(43) Pub. Date: Aug. 25, 2022

(54) ASPARTIC PROTEASE-TRIGGERED ANTIFUNGAL HYDROGELS

(71) Applicant: BROWN UNIVERSITY, Providence, RI (US)

(72) Inventors: Anita SHUKLA, Providence, RI (US); Noel VERA-GONZALEZ, San Juan, PR (US)

(21) Appl. No.: 17/682,782

(22) Filed: Feb. 28, 2022

Related U.S. Application Data
(63) Continuation of application No. 16/160,595, filed on Oct. 15, 2018, now abandoned.
(60) Provisional application No. 62/591,541, filed on Nov. 28, 2017.
(60) Provisional application No. 62/572,194, filed on Oct. 13, 2017.

(51) Int. Cl. A61K 47/42; A61K 47/69; A61K 31/7048; A61K 38/12; A61P 31/10; A61K 47/32; A61K 47/60; A61K 9/06; A61K 47/65

(52) U.S. Cl. CPC A61K 47/42; A61K 47/6903; A61K 31/7048; A61K 38/12; A61K 9/5026; A61K 47/32; A61K 47/60; A61K 9/06; A61K 47/65; A61P 31/10

(57) ABSTRACT

The present invention relates generally to antifungal hydrogels to locally deliver antifungal drugs. Specifically, the present invention provides aspartic protease-triggered antifungal hydrogels to locally deliver antifungal drugs that specifically respond to aspartic proteases secreted by virulent, pathogenic Candida.

### CROSS-REFERENCE TO RELATED APPLICATIONS

The present application is a continuation of U.S. patent application Ser. No. 16/160,595 filed Oct. 15, 2018 which claims the benefit of priority of U.S. Provisional Patent Application No. 62/572,194 filed Oct. 13, 2017 and U.S. Provisional Patent Application No. 62/591,541 filed Nov. 28, 2017, each of which are incorporated herein by reference in their entirety.

### GOVERNMENT RIGHTS

This invention was made with government support under grant number 1644760 awarded by the National Science Foundation and grant numbers N00014-14-1-0798 and N00014-17-1-2651 awarded by the Office of Naval Research. The government has certain rights in the invention.

### SEQUENCE LISTING

The instant application contains a Sequence Listing which has been filed electronically in ASCII format and is hereby incorporated by reference in its entirety. Said ASCII copy, created on Jan. 30, 2019, is named 405505-531001US_SL.txt and is 6,014 bytes in size.

### FIELD OF THE INVENTION

The present disclosure relates to antifungal hydrogels to locally deliver antifungal drugs. Specifically, the present disclosure relates to aspartic protease-triggered antifungal hydrogels to locally deliver antifungal drugs that specifically respond to aspartic proteases secreted by virulent, pathogenic Candida.

### BACKGROUND OF THE INVENTION

The polymorphic fungus Candida albicans is a commensal organism that colonizes the gastrointestinal tract, vagina and some cutaneous areas of the majority of healthy humans. However, under certain conditions the fungus is able to cause a variety of infections, ranging from mucosal to life-threatening invasive candidiasis. Candida is the most common cause of fungal infections, responsible for 46,000 infections per year in the United States.¹ Although C. albicans continues to be the most common cause of various forms of candidaisis, there are more than 20 species of Candida yeasts that can cause infection in humans.²⁻⁴ Invasive disease is associated with billions of dollars each year in healthcare costs and a mortality rate estimated at about 40%.⁵⁻⁶

Most of the common localized fungal infections are caused by Candida spp. The intensive use of antibiotics made these fungi more drug resistant and a clinical problem. Antimicrobial resistance of Candida is a growing threat that increases the severity of these infections. Some Candida strains are increasingly resistant to first-line and second-line antifungal treatment agents. Recent data demonstrate a marked shift among infections towards Candida species with increased resistance to antifungal drugs including azoles and echinocandins.^{2,7-8} For example, there are currently only four main classes of antifungal drugs and certain Candida albicans strains already show resistance to three of these four drug classes. A recently discovered strain, Candida auris, is resistant to all four drug classes. As a result, it is imperative to prevent further resistance from developing by using antifungals to treat serious infections only when the pathogenic phenotype is present in the infection site and limit the treatment to that infection site.

Accordingly, there remains a need for drug delivery systems that limit exposure to antimicrobials to the site of fungal infection and is triggered to selectively deliver the antifungal drug only in the presence of a virulent, pathogenic Candida fungal infection, thus helping prevent further development of resistance to antifungals and reduce off-site antifungal toxicity.

### BRIEF SUMMARY OF THE INVENTION

The present invention provides a biocompatible hydrogel system that is triggered to selectively release an antifungal drug in the presence of a virulent, pathogenic Candida fungal infection. The hydrogel system has (1) a plurality of cross-linkers connecting backbone components of the hydrogel, wherein the cross-linkers comprise a peptide sequence that is selectively cleaved by aspartic proteases (Saps) secreted by virulent, pathogenic Candida; and (2) an anti-fungal therapeutic agent encapsulated within the hydrogel. Virulent, pathogenic Candida include, but are not limited to, Candida albicans, Candida tropicalis, and Candida parapsilosis.

By incorporating a degradable peptide sequence into the hydrogel backbone that responds specifically to Saps secreted by virulent, pathogenic Candida, the biocompatible hydrogel system of the present invention can be utilized to deliver anti-fungal therapeutics and/or antibacterial therapeutics to a localized site within a patient. This allows for the selective delivery of the anti-fungal therapeutic and/or antibacterial therapeutic agent to a specified target within the patient because the Saps secreted by virulent, pathogenic Candida will cause degradation of the hydrogel backbone which will result in release of the anti-fungal therapeutic and/or antibacterial therapeutic agent.

Any peptide sequence containing linking group that is capable of being degraded by the secreted Saps by virulent, pathogenic Candida can be utilized. In some embodiments, the peptide is at least eight amino acids in length. While the peptide has no maximum length, so long as it is degradable by the desired enzyme, in certain embodiments the peptide is up to 8, 10, 20, 30, 50 or 100 amino acids in length. Some peptides are at least 5 or 10 amino acids in length. Each of these upper and lower limits are intended to be combinable to reflect some preferred peptide lengths. Examples of suitable peptides that can be degraded by Saps secreted by virulent, pathogenic Candida include at least one peptide comprising, consisting essentially of, or consisting of the following amino acid sequences: LRF(p-NO₂)↓FLAPK (SEQ ID NO: 1) (“LFFK”), LRFFLAPK (SEQ ID NO: 2, LRF(p-NO₂)↓FKAPK (SEQ ID NO: 3, LRFFKAPK (SEQ ID NO: 4), LRF(p-NO₂)↓FAAPK (SEQ ID NO: 5). LRFFAAPK (SEQ ID NO: K LRF(p-NO₂)↓FDAPK (SEQ ID NO: 7), LRFFDAPK (SEQ ID NO: 8), LRF(p-NO₂)↓FRAPK (SEQ ID NO: 9), LRFFRAPK (SEQ ID NO: 10), LRF(p-NO₂)↓FKDPK (SEQ ID NO: 11), LRFFKDPK (SEQ ID NO: 12), LRF(p-NO₂)↓FKRPK (SEQ ID NO: 13), LRFFKRPK (SEQ ID NO: 14), LRF(p-NO₂)↓FEIPK (SEQ ID NO: 15), LRFFEIPK (SEQ ID NO: 16), LAF(p-NO₂)↓FEAPK (SEQ ID NO: 17), LAFFEAPK (SEQ ID NO: 18), VFILWRTE (SEQ ID NO: 19), and/or TFSYnRWPK (SEQ ID NO: 20).

In certain aspects, suitable peptides that can be degraded by Saps secreted by virulent, pathogenic Candida include at least one peptide comprising, consisting essentially of, or consisting of the following amino acid sequences: LRF(p-NO₂)↓FLAPK (SEQ ID NO: 1) (“LFFK”), LRFFLAPK (SEQ ID NO: 2), LRF(p-NO₂)↓FKAPK (SEQ ID NO: 3). LRFFKAPK (SEQ ID NO: 4), LRF(p-NO₂)↓FAAPK (SEQ ID NO: 5), LRFFAAPK (SEQ ID NO: 6). LRF(p-NO₂)↓FDAPK (SEQ ID NO: 7), LRFFDAPK (SEQ ID NO: 8), LRF(p-NO₂)↓FRAPK (SEQ ID NO: 9, LRFFRAPK (SEQ ID NO: 10), LRF(p-NO₂)↓FKDPK (SEQ ID NO: 11), LRFFKDPK (SEQ ID NO: 12), LRF(p-NO₂)↓FKRPK (SEQ ID NO: 13), LRFFKRPK (SEQ ID NO: 14), LRF(p-NO₂)↓FEIPK (SEQ ID NO: 15), LRFFEIPK (SEQ ID NO: 16), LAF(p-NO₂)↓FEAPK (SEQ ID NO: 17), LAFFEAPK (SEQ ID NO: 18), LAFFEAPK (SEQ ID NO: 18), VFILWRTE (SEQ ID NO: 19), TFSYnRWPK (SEQ ID NO: 20), and/or peptides that are at least about 50% 60%, 65%, 70%, 75%, 80%, 85%, 90%, 91%, 92%, 93%, 94%, 95%, 96%, 97%, 98%, or 99% homologous thereto.

In certain aspects, the phenylalanines and 4-nitro-phenylalanines of the above-disclosed peptides that can be degraded by Saps secreted by virulent, pathogenic Candida are considered necessary amino acid residues to maintain the peptides' function. In other aspects, it is believed that the phenylalanines and 4-nitro-phenylalanines of the above-disclosed peptides that can be degraded by Saps secreted by virulent, pathogenic Candida can be substituted conservatively, and such amino acid substitutions would not significantly diminish their ability to be selectively cleaved by Saps secreted by virulent, pathogenic Candida. In certain aspects, it is believed that amino acids other than the phenylalanines and 4-nitro-phenylalanines of the above-disclosed peptides that can be degraded by Saps secreted by virulent, pathogenic Candida may be substituted either conservatively or non-conservatively, and such amino acid substitutions would not significantly diminish their ability to be selectively cleaved by Saps secreted by virulent, pathogenic Candida. In other aspects, it is believed that amino acids other than the phenylalanines and 4-nitro-phenylalanines of the above-disclosed peptides that can be degraded by Saps secreted by virulent, pathogenic Candida may be substituted conservatively, and such amino acid substitutions would not significantly diminish their ability to be selectively cleaved by Saps secreted by virulent, pathogenic Candida.

Hydrogels are well known in the art and are generally formed by the reaction of a macromer having a biocompatible backbone with a cross-linking agent. It is anticipated that any covalently cross-linked hydrogel may be utilized. Types of materials that could be used for this purpose include crosslinked synthetic hydrogels that are based on molecules like hyaluronic acid or polyethylene glycols. Suitable hydrogels also include those constructed using polyesters, polyurethanes, polysaccharides, proteins, and combinations thereof. Polyesters, poly(ethylene oxide) (PEO), proteins and the like are also suitable polymeric materials that can be used as a polymeric component of the hydrogel.

In certain aspects, macromers include polyethylene glycols, hyaluronic acid, polyesters, polyurethanes, polysaccharides, and/or proteins. In certain aspects, polymeric components include polyesters, poly(ethylene oxide), proteins, gellan gum, alginate, and/or pectin. A partial listing of polysaccharides that are useful in the claimed invention includes hyaluronic acid, amylase, amylopectin, glycogen, cellulose, heparin, agarose, alginate, and the like. In some embodiments, hyaluronic acid or any combination thereof is particularly suitable for use in the instant invention.

The macromers may include a range of polymerizing moieties, such as acrylates, methacrylates, and the like. In some embodiments, the polymerizing moiety includes a carbon-carbon double or triple bond. The moiety is suitably polymerized by photopolymerization, by free radical-initiation, or by other methods of polymerization known to those of skill in the art.

The responsive peptide moieties (the suitable peptides that can be degraded by Saps secreted by virulent, pathogenic Candida) can be incorporated into the cross-linkers by reaction of active hydrogen atoms. In some embodiments, the active hydrogen atoms can be part of hydroxy, thiol, or amine groups (including hydrazine). In some embodiments, the peptide can be incorporated as crosslinks through the addition reaction of thiols in cysteines in the peptides with acrylate or methacrylates, vinyl sulfones, or maleimides on these molecules. In certain aspects, the responsive peptide moieties are conjugated to PEG-acrylate functionalized with succinimidyl valerate under basic conditions. However, it should be understood that PEG-acrylate could be substituted with other polymers as long as the other polymers are covalently linked by the response peptide moiety(ies) and its cleavage results in hydrogel degradation.

Any useful anti-fungal and/or anti-bacterial therapeutic agent can be utilized with the presently-disclosed hydrogel system. In some embodiments, antifungal drugs include, but are not limited to, one or more of the following antifungal drugs: allylamines (such as amorolfine (Locery®), butenafine (Lotrimin®, Mentax®), naftifine (Naftin®), terbinafine (Lamisil®)); imidazoles (such as binfonazole (Canespor®), butoconazole (Femstat®, Gynazole®), clotrimazole (Canesten®, Clocreme®, Cruex®, Desenex®, Femcare®, Fungoid®, Gyne-Lotrimin®, Gynix®, Lotrimin, Mycelex®, Pedesil®, Trivagizole®), econazole (Ecoza®, Spectazole®), fenticonazole (Lomexin®, Gynoxin®), isoconazole (Icaden®, Travogen®), ketoconazole (Nizoral®, Extina®, Ketodan, Kuric®, Xolegel®), luliconazole (Luzu), miconazole (Cavilon®, Cruex®, Desenex®, Fungoid®, Lotrimin®, Micatin®, Monistat®, Oravig, Ting®, Vagistat®, Zeasorb®), omoconazole (Fongamil®), oxiconazole (Oxistat®), sertaconazole (Ertaczo®), sulconazole (Exelderm®), tioconazole (Monistat®, Vagistat®), terconazole (Terazol®, Terconazole®, Zazole®); triazoles (such as albaconazole, efinaconazole (Jublia®), fluconazole (Diflucan®), isavuconazole or isavuconazonium (Cresemba®), itraconazole (Sporanox®, Onmel®), posaconazole (Noxafil®), ravuconazole, terconazole (Terazol®), voriconazole (Vfend®)), arylguanidines or thiazoles (such as abafungin (Abasol®)); polyenes (such as Amphotericin B (Fungizone, Fungilin®, AmBisome®), nystatin (Nilstat®), natamycin (pimaricin), trichomycin (hachimycin); echinocandins (such as anidulafungin (Eraxis®), caspofungin (Cancidas®), micafungin (Mycamine®); thiocarbamates (such as tolnaftate (Tinactin®, Aftate®, Breezee®, Ting®)); antimetabolites (such as flucytosine (Ancobon®)); benzylamines (such as butenafine (Mentax®, Lotrimin®); and other antifungals such as griseofulvin (Gris-PEG®, Grifulvin®, Grisactin®); ciclopirox (Ciclodan®, Loprox®, Penlac®, Loprox®); selenium sulfide (Selsun®, Exsel®); tavaborole (Kerydin®); among others.

Antimicrobial peptides can also be encapsulated or covalently tethered to the hydrogel backbone. Furthermore, other therapeutics such as antibiotics, endothelial growth factors, hormones, or clotting factors can be encapsulated to also aid in wound healing. In some embodiments, the therapeutic agent(s) may be directly encapsulated during the gelation process by mixing the molecule with the pre-cursor solutions or covalently tethered to the hydrogel backbone using chemistry described previously.

In certain aspects, the fabrication process for forming the instant hydrogels poses advantages to other currently used methods of forming hydrogels which involve harsh chemicals for the polymerization of hydrogels. In certain aspects, the fabrication process for forming the instant hydrogels involves using white light and an aqueous buffer as the solvent. This method allows for controlling the shape and rigidity of the instant hydrogels with ease, and can even be used to photopolymerize the hydrogels in situ. It will be readily understood that the instant hydrogel system can support the delivery of different antifungal agents as well as readily be modified to work against other fungal strains.

The present invention further provides a process for delivery of an anti-fungal to the extracellular matrix of target tissue. In certain aspects, the process comprises

(1) administering a biocompatible hydrogel having a plurality of cross-linkers connecting backbone components of the hydrogel, wherein the cross-linkers comprise a peptide sequence that is selectively cleaved by aspartic proteases secreted by virulent, pathogenic Candida; and an anti-fungal therapeutic agent encapsulated within the hydrogel; and (2) allowing the hydrogel to contact the aspartic proteases secreted by virulent, pathogenic Candida in the extracellular matrix of the target tissue, wherein the contact results in the release of at least a portion of the anti-fungal therapeutic agent.

The present invention further provides a method of treating a virulent, pathogenic Candida infection in a subject by administering a biocompatible hydrogel that comprises a plurality of cross-linkers connecting backbone components of the hydrogel and an anti-fungal therapeutic agent encapsulated within the hydrogel. The hydrogel is cross-linked utilizing a cross-linker comprising a peptide sequence that is selectively cleaved by aspartic proteases secreted by virulent, pathogenic Candida. Once the hydrogel comes in contact with aspartic proteases secreted by virulent, pathogenic Candida, at least a portion of the anti-fungal therapeutic agent is released into the site of infection.

The responsive hydrogel material can also be used to coat a wide range of medical device surfaces to prevent biofilm formation and subsequent medical device related infections. Different surfaces, including but not limited to, glass, plastic, and metals can be etched and functionalized with 10% 3(trimethoxysilyl)propyl methacrylate (TMSPMA) in acetone. This allows covalent attachment of Ac-PEG-responsive peptide-PEG-Ac conjugates to the surface of the material using the photopolymerization technique described above, forming thin hydrogel coatings. As such, the present invention further provides a method of preventing a virulent, pathogenic Candida infection in a subject by applying a biocompatible hydrogel to a surface subject to exposure to and contamination with the virulent, pathogenic Candida. The biocompatible hydrogel comprises a plurality of cross-linkers connecting backbone components of the hydrogel and an anti-fungal therapeutic agent encapsulated within the hydrogel. The hydrogel is cross-linked utilizing a cross-linker comprising a peptide sequence that is selectively cleaved by aspartic proteases secreted by virulent, pathogenic Candida. When the treated surface is contaminated with a virulent, pathogenic Candida, the hydrogel comes in contact with aspartic proteases secreted by virulent, pathogenic Candida, at least a portion of the anti-fungal therapeutic agent thus preventing an infection.

Other implementations are also described and recited herein.

### BRIEF DESCRIPTION OF THE DRAWINGS

FIG. 1 is a schematic representation of the fungal enzyme-responsive hydrogel system exhibiting a triggered release of antifungal therapies.

FIG. 2 depicts the structure of LFFK. FIG. 2 discloses SEQ ID NO: 1.

FIG. 3 depicts the characterization of the LFFK peptide and conjugate. Panel A depicts LFFK peptide LC-MS results showing the C-18 liquid chromatogram (top) and the mass spectrometry analysis (bottom). Panel B depicts the size exclusion chromatography of a representative conjugate and PEG starting material.

FIG. 4 illustrates the successful degradation of the LFFK peptide in the presence of aspartic proteases. FIG. 4 discloses the degradation products of DEQ ID NO: 1 upon cleavage by Saps. FIG. 4 discloses SEQ ID NOS 1 and 23, respectively, in order of appearance.

FIG. 5 depicts the characterization of the PEG-LFFK-PEG conjugate using MALDI-TOF and size exclusion chromatography.

FIG. 6 illustrates the successful degradation of the PEG-LFFK-PEG conjugate in the presence of aspartic protease from C. albicans 28366.

FIG. 7 illustrates the fabrication of the fungal enzyme-responsive hydrogel system. Panel A depicts a hydrogel photopolymerization scheme. Panel B depicts bulk hydrogel (left) and microsphere (right) containing 10% Acryl-PEG-LFFK-PEG-Acryl and anidulafungin.

FIG. 8 illustrates the degradation of a responsive hydrogel system on infected agar over time.

FIG. 9 illustrates the successful degradation of 10% PK hydrogels loaded with 0.5% (w/v) anidulafungin when exposed to a 2 mg/mL concentration of C. albicans secreted aspartic proteases.

FIG. 10 illustrates the activity assay of hydrogels encapsulating liposomal amphotericin B (AmBisome) fungicidal. Panel A depicts schematically the in vitro assay. The hydrogels are cultured with Candida in Sap inducing media and after 24-hour periods, part of the culture solution is removed, diluted and plated after which the colony forming units are counted and compared to a control culture. Panel B depicts the fungal survival after exposure to the responsive hydrogels. Panel C depicts the proteolytic activity of culture.

FIG. 11 illustrates the stability of PK hydrogels in the presence of penicillinases and lipases (upper panel) and that non-responsive PEG hydrogels remained intact in the presence of aspartic proteases for at least 24 hours.

FIG. 12 illustrates the effects of polymer concentration on drug release and hydrogel mechanical properties. Panel A depicts the hydrogel cumulative drug release profile in enzymes secreted by Candida (Saps); * p<0.01 between both conditions, two-tailed unpaired t-test. Panel B depicts the hydrogel mechanical properties; * p<0.01, two-tailed unpaired t-test, n=3.

FIG. 13 illustrates the covalent conjugation of the antifungal drug amphotericin B with acrylated PEG (Panel A); the matrix-assisted laser desorption/ionization—time of flight mass spectroscopy chromatogram confirming drug-PEG conjugation noting the shift in molecular weight (Panel B); the efficacy of drug-PEG conjugate in inhibiting Candida growth at 32 μg/mL (Panel C); and the efficacy of responsive hydrogels (with LFFK peptide) bearing covalently linked drug-PEG (Panel D).

FIGS. 14A-C illustrate peptide catalytic efficiency modifications. FIG. 14A depicts peptide molecular structures. FIG. 14B depicts LC-MS chromatogram of synthesized peptides. FIG. 14C depicts the degradation of hydrogels bearing the different peptides in the presence of Saps; * p<0.05, two-tailed unpaired t-test, n=3.

### DETAILED DESCRIPTION OF THE INVENTION

It is to be appreciated that certain aspects, modes, embodiments, variations and features of the invention are described below in various levels of detail in order to provide a substantial understanding of the present invention.

The following description of particular aspect(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only. While the compositions or processes are described as using specific materials or an order of individual steps, it is appreciated that materials or steps may be interchangeable such that the description of the invention may include multiple parts or steps arranged in many ways as is readily appreciated by one of skill in the art.

### Definitions

The definitions of certain terms as used in this specification and the appended claims are provided below. Unless defined otherwise, all technical and scientific terms used herein generally have the same meaning as commonly understood by one of ordinary skill in the art to which this invention belongs.

As used in this specification and the appended claims, the singular forms “a,” “an” and “the” include plural referents unless the content clearly dictates otherwise. For example, reference to “a cell” includes a combination of two or more cells, and the like.

The term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B: B or C; A and C; A and B; B and C; A (alone); B (alone); and C (alone).

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, reference to values stated in ranges include each and every value within that range.

The term “subject” refers to a mammal, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), laboratory animals (e.g., mice, rats, rabbits, etc.), but are not so limited. Subjects include human subjects. The human subject may be a pediatric, adult, or a geriatric subject. The human subject may be of either sex.

The terms “effective amount” and “therapeutically-effective amount” include an amount sufficient to prevent or ameliorate a manifestation of disease or medical condition, such as an infection. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the disease and on the characteristics of the subject, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds.

As used herein, the term “biocompatible” means that the components, in addition to the therapeutic agent, comprising the hydrogel system, are suitable for administration to the patient being treated in accordance with the present invention.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. The names of the natural amino acids are abbreviated herein in accordance with the recommendations of IUPAC-IUB.

The terms “identity” and “identical” refer to a degree of identity between sequences, there may be partial identity or complete identity. A partially identical sequence is one that is less than 100% identical to another sequence. Partially identical sequences may have an overall identity of at least 70% or at least 75%, at least 80% or at least 85%, or at least 90% or at least 95%.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

The terms “modulation” and “modulate” as used herein refer to a change or an alteration in a biological activity. Modulation includes, but is not limited to, stimulating an activity or inhibiting an activity. Modulation may be an increase or a decrease in activity, a change in binding characteristics, or any other change in the biological, functional, or immunological properties associated with the activity of a protein, a pathway, a system, or other biological targets of interest.

The terms “treating” or “treatment” or “to treat” or “alleviating” or “to alleviate” refer to both (1) therapeutic measures that cure, slow down, lessen symptoms of, and/or halt progression of a diagnosed fungal infection and (2) prophylactic or preventative measures that prevent or slow the development of a fungal infection.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

### Antifungal Drug Resistance

Antifungal drugs save lives by treating dangerous fungal infections. Unfortunately, fungi can develop the ability to defeat the drugs designed to kill them. Antifungal resistance is especially a concern for patients with invasive infections like those caused by the fungus Candida, a yeast, which can cause serious health problems, including disability and death. Patients can get fungal infections while receiving care for something else in a healthcare facility. For example, Candida is a leading cause of healthcare-associated bloodstream infections in US hospitals.⁹ These infections are also costly for patients and healthcare facilities. Each case of Candida bloodstream infection (also known as candidemia) is estimated to result in an additional 3 to 13 days of hospitalization and $6,000 to $29,000 in healthcare costs.¹⁰

Antifungal resistance is a particular problem with Candida infections. Some types of Candida are increasingly resistant to the first-line and second-line antifungal medications, such as fluconazole and the echinocandins (anidulafungin, caspofungin, and micafungin). About 7% of all Candida bloodstream isolates tested at CDC are resistant to fluconazole. More than 70% of these resistant isolates are the species Candida glabrata or Candida krusei.^{11,12}

Multidrug-resistant Candida infections (those that are resistant to both fluconazole and an echinocandin) have very few remaining treatment options. The primary treatment option is Amphotericin B, a drug that can be toxic for patients who are already very sick. Not surprisingly, there is growing evidence to suggest that patients who have drug-resistant candidemia are less likely to survive than patients who have candidemia that can be treated by antifungal medications.^{13,14} Emerging antifungal resistance has been identified in species like Candida auris.¹⁵ Isolates of C. auris sent to CDC are almost all resistant to fluconazole, and up to one-third are resistant to amphotericin B, usually reserved as a last-resort treatment.¹⁶ Most C. auris isolates are susceptible to echinocandins. However, echinocandin resistance can develop while the patient is being treated. C. auris is also a concerning public health issue because it is difficult to identify with standard laboratory methods and because it spreads easily in healthcare settings, such as hospitals and long-term care facilities. Accordingly, it is imperative to prevent further resistance from developing.

### Aspartic Protease Triggered Anti-Fungal Hydrogel System

The presently-disclosed data relates to a responsive hydrogel system to combat skin wound fungal infections by incorporating a degradable peptide sequence that responds specifically to aspartic proteases (Saps) secreted by virulent, pathogenic Candida into the hydrogel backbone. The presently-disclosed data demonstrates that the responsive hydrogel system provides for a controlled drug release mechanism. Currently, no such system exists to treat fungal infections. The instantly-disclosed hydrogels degrade and release their loaded drug only in the presence of virulent, pathogenic Candida, for example, but not limited to, Candida albicans. Otherwise, the hydrogels remain intact in a culture of non-pathogenic Candida. Making the distinction between virulent, pathogenic and non-pathogenic fungi is important in preventing antifungal drug resistance. As described above, there are currently only four main classes of antifungal drugs and certain Candida albicans strains already show resistance to three of these four drug classes. A recently discovered strain, Candida auris, is resistant to all four drug classes. As a result, it is imperative to use antifungals to treat serious infections only when the virulent, pathogenic phenotype is present in the infection site. The instantly-disclosed hydrogels do just that, as they respond to Saps, which are Candida virulence markers.

Additionally, in certain aspects, the fabrication process for forming the instant hydrogels poses advantages to other currently used methods of forming hydrogels which involve harsh chemicals for the polymerization of hydrogels. In certain aspects, the fabrication process for forming the instant hydrogels involves using white light and an aqueous buffer as the solvent. This method allows for controlling the shape and rigidity of the instant hydrogels with ease, and can even be used to photopolymerize the hydrogels in situ. It will be readily understood that the instant hydrogel system can support the delivery of different antifungal agents as well as readily be modified to work against other fungal strains.

In certain aspects, the present disclosure relates to a biocompatible hydrogel. In certain aspects, the biocompatible hydrogel comprises a plurality of cross-linkers connecting backbone components of said hydrogel, wherein said hydrogel is cross-linked utilizing a cross-linker comprising a peptide sequence that is selectively cleaved by aspartic proteases secreted by virulent, pathogenic Candida. Further, the biocompatible hydrogel comprises an anti-fungal therapeutic agent, said anti-fungal therapeutic agent encapsulated within said hydrogel. Thus, the instant hydrogels incorporating a degradable peptide sequence that responds specifically to aspartic proteases (Saps) secreted by virulent, pathogenic Candida into the hydrogel backbone can be utilized in the delivery of anti-fungal therapeutics and/or antibacterial therapeutics to a localized site within a patient. In certain aspects of the present disclosure, the cross-linkers of the instant hydrogels comprise a peptide sequence that is degradable by aspartic proteases (Saps) secreted by virulent, pathogenic Candida. This allows for the selective delivery of the anti-fungal therapeutic and/or antibacterial therapeutic agent to a specified target within the patient because the Saps secreted by virulent, pathogenic Candida will cause degradation of the hydrogel backbone which will result in release of the anti-fungal therapeutic and/or antibacterial therapeutic agent. As such, certain aspects of the present disclosure relate to a process for delivery of an anti-fungal to the extracellular matrix of target tissue. In certain aspects, the process comprises administering a biocompatible hydrogel, said hydrogel comprising a plurality of cross-linkers connecting backbone components of said hydrogel, wherein said hydrogel is cross-linked utilizing a cross-linker comprising a peptide sequence that is selectively cleaved by aspartic proteases secreted by virulent, pathogenic Candida. Further, the biocompatible hydrogel comprises an anti-fungal therapeutic agent, with said anti-fungal therapeutic agent encapsulated within said hydrogel. The process further comprises allowing said hydrogel to contact aspartic proteases secreted by virulent, pathogenic Candida in said extracellular matrix of the target tissue, said contact resulting in the release of at least a portion of said anti-fungal therapeutic agent.

Any peptide sequence containing linking group that is capable of being degraded by the secreted Saps by virulent, pathogenic Candida can be utilized. Virulent, pathogenic Candida include, but are not limited to, Candida albicans, Candida tropicalis, and Candida parapsilosis. In some embodiments, the peptide is at least eight units in length. While the peptide has no maximum length, so long as it is degradable by the desired enzyme, in certain embodiments the peptide is up to 8, 10, 20, 30, 50,100 or 200 units in length. Some peptides are at least 5 or 10 units in length. Each of these upper and lower limits are intended to be combinable to reflect some preferred peptide lengths. Examples of suitable peptides that can be degraded by Saps secreted by virulent, pathogenic Candida include at least one peptide comprising, consisting essentially of, or consisting of the following amino acid sequences: LRF(p-NO₂)↓FLAPK (SEQ ID NO: 1) (“LFFK”), LRFFLAPK (SEQ ID NO: 2), LRF(p-NO₂)↓FKAPK (SEQ ID NO: 3), LRFFKAPK (SEQ ID NO: 4), LRF(p-NO₂)↓FAAPK (SEQ ID NO: 5), LRFFAAPK (SEQ ID NO: 6), LRF(p-NO₂)↓FDAPK (SEQ ID NO: 7), LRFFDAPK (SEQ ID NQ: 8), LRF(p-NO₂)↓FRAPK (SEQ ID NO: 9), LRFFRAPK (SEQ ID NO: 10), LRF(p-NO₂)↓FKDPK (SEQ ID NO: 11), LRFFKDPK (SEQ ID NO: 12), LRF(p-NO₂)↓FKRPK (SEQ ID NO: 13), LRFFKRPK (SEQ ID NO: 14), LRF(p-NO₂)↓FEIPK (SEQ ID NO: 15), LRFFEIPK (SEQ ID NO: 16), LAF(p-NO₂)↓FEAPK (SEQ ID NO: 17), LAFFEAPK (SEQ ID NO: 18), VFILWRTE (SEQ ID NO: 19), and/or TFSYnRWPK (SEQ ID NO: 20). The phrase “consisting essentially of,” as used herein, is intended to mean that additional amino acids or other residues may be present at either terminus of the peptide and/or on a side chain provided they do not substantially impair the activity of the peptide to be selectively degraded by Saps secreted by virulent, pathogenic Candida.

In certain aspects, suitable peptides that can be degraded by Saps secreted by virulent, pathogenic Candida include at least one peptide comprising, consisting essentially of, or consisting of the following amino acid sequences: LRF(p-NO₂)↓FLAPK (“LFFK”) LRF(p-NO₂)↓FLAPK (SEQ ID NO: 1) (“LFFK”), LRFFLAPK (SEQ ID NO: 2), LRF(p-NO₂)↓FKAPK (SEQ ID NO: 3), LRFFKAPK (SEQ ID NO: 4), LRF(p-NO₂)↓FAAPK (SEQ ID NO: 5), LRFFAAPK (SEQ ID NO: 6), LRF(p-NO₂)↓FDAPK (SEQ ID NO: 7), LRFFDAPK (SEQ ID NO: 8, LRF(p-NO₂)↓FRAPK (SEQ ID NO: 9), LRFFRAPK (SEQ ID NO: 10), LRF(p-NO₂)↓FKDPK (SEQ ID NO: 11), LRFFKDPK (SEQ ID NO: 12), LRF(p-NO₂)↓FKRPK (SEQ ID NO: 13), LRFFKRPK (SEQ ID NO: 14), LRF(p-NO₂)↓FEIPK (SEQ ID NO: 15, LRFFEIPK (SEQ ID NO: 16), LAF(p-NO₂)↓FEAPK (SEQ ID NO: 17), LAFFEAPK (SEQ ID NO: 18), VFILWRTE (SEQ ID NO: 19), TFSYnRWPK (SEQ ID NO: 20) and/or peptides that are at least about 50% 60%, 65%, 70%, 75%, 80%, 85%, 90%, 91%, 92%, 93%, 94%, 95%, 96%, 97%, 98%, or 99% homologous thereto. Percent homology can be determined as is known in the art. For example, to determine the percent identity of two amino acid sequences, the sequences are aligned for optimal comparison purposes (e.g., gaps can be introduced in one or both of a first and a second amino acid sequence for optimal alignment and non-homologous sequences can be disregarded for comparison purposes). The amino acid residues at corresponding amino acid positions are then compared. When a position in the first sequence is occupied by the same amino acid residue as the corresponding position in the second sequence, then the molecules are identical at that position (as used herein amino acid “identity” is equivalent to amino acid “homology”). As is known in the art, the percent identity between the two sequences is a function of the number of identical positions shared by the sequences, taking into account the number of gaps and the length of each gap, which need to be introduced for optimal alignment of the two sequences. Sequence homology for polypeptides is typically measured using sequence analysis software.

When homologous is used in reference to peptides, it is recognized that residue positions that are not identical can often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are known to those of skill in the art. The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (1), Leucine (L), Methionine (M), Alanine (A), Valine (V), and 6) Phenylalanine (F), Tyrosine (Y), Tryptophan (W).

In certain aspects, the phenylalanines and 4-nitro-phenylalanines of the above-disclosed peptides that can be degraded by Saps secreted by virulent, pathogenic Candida are considered necessary amino acid residues to maintain the peptides' function. In other aspects, it is believed that the phenylalanines and 4-nitro-phenylalanines of the above-disclosed peptides that can be degraded by Saps secreted by virulent, pathogenic Candida can be substituted conservatively, and such amino acid substitutions would not significantly diminish their ability to be selectively cleaved by Saps secreted by virulent, pathogenic Candida. In certain aspects, it is believed that amino acids other than the phenylalanines and 4-nitro-phenylalanines of the above-disclosed peptides that can be degraded by Saps secreted by virulent, pathogenic Candida may be substituted either conservatively or non-conservatively, and such amino acid substitutions would not significantly diminish their ability to be selectively cleaved by Saps secreted by virulent, pathogenic Candida. In other aspects, it is believed that amino acids other than the phenylalanines and 4-nitro-phenylalanines of the above-disclosed peptides that can be degraded by Saps secreted by virulent, pathogenic Candida may be substituted conservatively, and such amino acid substitutions would not significantly diminish their ability to be selectively cleaved by Saps secreted by virulent, pathogenic Candida.

The degradation or breaking of the crosslinks formed by the peptides that can be degraded by Saps secreted by virulent, pathogenic Candida in the hydrogel alters the crosslinking density, which in turn alters the material properties (i.e., mechanics), which alters the diffusion of molecules through the hydrogel and hence delivery into the affected tissue. With such a hydrogel material, the release of the anti-fungal therapeutics and/or antibacterial therapeutics will be locally dependent on the level of Saps activity secreted by virulent, pathogenic Candida at those sites. For example, the instantly-disclosed data demonstrates that hydrogels formed with a peptide that can be degraded by Saps secreted by virulent, pathogenic Candida was preferentially cleaved by pepsin and the Saps between the two hydrophobic phenylalanines or between leucine and phenylalanine, as confirmed by HPLC-MS, resulting in hydrogel degradation. In the presence of pepsin, the hydrogels release 2.1±1.3% of the total loaded drug within one hour, 64.9±10% after two hours, and 88.4±1.02% after twenty-four hours. Non-peptide containing hydrogels (i.e., pure PEG hydrogels) remained intact in pepsin for at least 24 hours, releasing just 0.05±0.003% of the total loaded drug. In the presence of Saps, the hydrogels released 72.28±2.96% of the total loaded drug within one hour. Responsive hydrogels cultured in an acidic buffer lacking enzymes released only 0.43±1.01% of the total loaded drug in 24 hours. No hydrogel degradation was observed in HEPES buffered saline (HBS), indicating that these hydrogels are specifically responsive to aspartic proteases. Further, the hydrogels proved to be stable in lipases and penicillinases, releasing only 0.64±0.20% and 0.67±0.59% of the total loaded drug, respectively, after 24 hours. These are enzymes secreted by other microbes, suggesting minimal interference from their presence at an infection site.

Hydrogels are well known in the art and are generally formed by the reaction of a macromer having a biocompatible backbone with a cross-linking agent. It is anticipated that any covalently cross-linked hydrogel may be utilized. Types of materials that could be used for this purpose include crosslinked synthetic hydrogels that are based on molecules like hyaluronic acid or polyethylene glycols. Suitable hydrogels also include those constructed using polyesters, polyurethanes, polysaccharides, proteins, and combinations thereof. Polyesters, poly(ethylene oxide) (PEO), proteins and the like are also suitable polymeric materials that can be used as a polymeric component of the hydrogel. General synthetic methods for making hydrogels can be found, for example in Burdick, et al.¹⁷

In certain aspects, macromers include polyethylene glycols, hyaluronic acid, polyesters, polyurethanes, polysaccharides, and/or proteins. In certain aspects, polymeric components include polyesters, poly(ethylene oxide), proteins, gellan gum, alginate, and/or pectin. A partial listing of polysaccharides that are useful in the claimed invention includes hyaluronic acid, amylase, amylopectin, glycogen, cellulose, heparin, agarose, alginate, and the like. In some embodiments, hyaluronic acid or any combination thereof is particularly suitable for use in the instant invention.

The macromers may include a range of polymerizing moieties, such as acrylates, methacrylates, and the like. In some embodiments, the polymerizing moiety includes a carbon-carbon double or triple bond. The moiety is suitably polymerized by photopolymerization, by free radical-initiation, or by other methods of polymerization known to those of skill in the art.

The responsive peptide moieties (the suitable peptides that can be degraded by Saps secreted by virulent, pathogenic Candida) can be incorporated into the cross-linkers by reaction of active hydrogen atoms. In some embodiments, the active hydrogen atoms can be part of hydroxy, thiol, or amine groups (including hydrazine). In some embodiments, the peptide can be incorporated as crosslinks through the addition reaction of thiols in cysteines in the peptides with acrylate or methacrylates, vinyl sulfones, or maleimides on these molecules.

Polyethylene glycol (PEG) is a non-toxic bioinert polymer that is highly soluble in water. It has been used extensively in drug delivery applications as well as in the fabrication of hydrogels with high oxygen permeability, which facilitates wound healing. In certain aspects, the responsive peptide moieties are conjugated to PEG-acrylate functionalized with succinimidyl valerate under basic conditions. However, it should be understood that PEG-acrylate could be substituted with other polymers as long as the other polymers are covalently linked by the response peptide moiety(ies) (as described previously) and its cleavage results in hydrogel degradation.

The instantly-disclosed hydrogel systems can have varying mesh sizes. Hydrogel mesh size will be calculated using the Peppas-Merrill model (adapted from the Flory-Rehner model) in conjunction with the Canal-Peppas model.¹⁸ Changes in polymer molecular weight and concentration can have an effect on the hydrogel's mesh size, mechanical properties, and subsequently, enzyme penetration and degradation rates. An increase in polymer concentration and photopolymerization times increases crosslinking density, which has been shown to reduce hydrolytic degradation rates, diffusivity, and gel swelling which in turn decrease hydrogel mesh size, but increase hydrogel stiffness and hydrophobicity. Hydrogels with a high polymeric concentration are expected to degrade at slower rates, preventing Sap enzyme diffusion into the interior of the hydrogel, as well as to reduce unwanted diffusion of drug out from the hydrogels. Higher polymeric concentrations are also expected to aid antifungal drug retention, owing to the fact that most antifungal drugs, such as anidulafungin, are hydrophobic. Hydrogel mesh size for the 10% (w/v) PEG-LFFK-PEG hydrogels tested should range between 6.3 to 10.8 nm as shown by other PEG-peptide-PEG hydrogels of similar MW prepared using the same photoinitiator, Eosin Y. Since mesh size is irregular partly due to polymer polydispersity and entanglements, the hydrogel mesh size of 10% (w/v) gels may allow Sap penetration to some extent. Increasing the ratio of non-responsive PEG in the hydrogel backbone can also slow down degradation rates. A fine balance must exist, however, since too much non-responsive PEG can render the hydrogel nondegradable. Thus, we can develop a highly tunable system that encapsulates and releases drug in the presence of Saps, whether present in low or high concentrations. Preliminary results show that 5% PEG-LFFK-PEG hydrogels that are cultured in buffer (no Saps) release 3.11±0.12% of their total loaded drug (anidulafungin) after just 3 hours because of passive diffusion. However, 10% PEG-LFFK-PEG hydrogels, which should have a smaller mesh size, released only 0.43±0.01% of their total loaded drug (anidulafungin) after 24 hours when cultured in buffer (no Saps). The minimal drug release from unperturbed hydrogels is due to the drug's (anidulafungin) hydrophobicity. PEG, being very hydrophilic, can repel the drug, allowing it to remain inside the hydrogel's pores.

The responsive hydrogel material of the present invention can also be used to coat a wide range of medical device surfaces to prevent biofilm formation and subsequent medical device related infections. Different surfaces, including but not limited to, glass, plastic, and metals can be etched and functionalized with 10% 3(trimethoxysilyl)propyl methacrylate (TMSPMA) in acetone. This allows covalent attachment of Ac-PEG-responsive peptide-PEG-Ac conjugates to the surface of the material using the photopolymerization technique described above, forming thin hydrogel coatings.

### Antifungal and Anti-Bacterial Therapeutic Agents Useful in the Hydrogel System

Any useful anti-fungal and/or anti-bacterial therapeutic agent can be utilized with the presently-disclosed hydrogel system. In some embodiments, antifungal drugs include, but are not limited to, allylamines, azoles (including imidazole, triazole, and arylguanidine derivatives), polyenes, echinocandins, thiocarbamates, antimetabolites, benzylamines and other antifungal drugs such as griseofulvin, ciclopirox, selenium sulfide, tavaborole, amongst others.

Allylamines are synthetic antifungals with activity against a wide range of dermatophytes. Allylamines act via inhibition of the squalene epoxidase formation, which blocks the synthesis of ergosterol. Allylamines (with the exception of terbinafine) are used as topical treatments. Oral terbinafine is extensively used for the treatment of onychomycosis (fungal infection of the nail). It acts at an earlier stage by inhibiting the formation of squalene epoxide, a precursor of lanosterol. Oral terbinafine is the first choice for treating infections of fingernails and toenails. Examples of allylamines include amorolfine (Locery®), butenafine (Lotrimin®, Mentax®), naftifine (Naftin®), terbinafine (Lamisil®).

Azoles (imidazole and triazole derivatives) are a large group of synthetic antifungal agents. Azoles are essentially fungistatic, and have a relatively broad antifungal spectrum. The azole antifungals have many drug-drug interactions because of their interference with cytochrome P-450 enzymes. Imidazoles are considered first-line agents for most dermatophyte infections. Topical formulations are widely used for the treatment of superficial fungal infections and vaginal candidiasis. Imidazoles are very toxic when taken orally, so they are available only as topical formulations. Examples of imidazoles include binfonazole (Canespor®), butoconazole (Femstat®, Gynazole®), clotrimazole (Canesten®, Clocreme®, Cruex®, Desenex®, Femcare®, Fungoid®, Gyne-Lotrimin®, Gynix®, Lotrimin, Mycelex®, Pedesil®, Trivagizole®), econazole (Ecoza®, Spectazole®), fenticonazole (Lomexin®, Gynoxin®), isoconazole (Icaden®, Travogen®), ketoconazole (Nizoral®, Extina®, Ketodan, Kuric®, Xolegel®), luliconazole (Luzu®), miconazole (Cavilon®, Cruex®, Desenex®, Fungoid®, Lotrimin®, Micatin®, Monistat®, Oravig, Ting®, Vagistat®, Zeasorb®), omoconazole (Fongamil®), oxiconazole (Oxistat®), sertaconazole (Ertaczo®), sulconazole (Exelderm®), tioconazole (Monistat®, Vagistat®), terconazole (Terazol®, Terconazole®, Zazole®). Triazoles are generally used for prophylaxis and treatment of invasive fungal infections and systemic mycosis. Examples of triazoles include albaconazole, efinaconazole (Jublia®), fluconazole (Diflucan®), isavuconazole or isavuconazonium (Cresemba®), itraconazole (Sporanox®, Onmel®), posaconazole (Noxafil®), ravuconazole, terconazole (Terazol®), voriconazole (Vfend®). Arylguanidines or thiazoles are a novel class of synthetic antifungal drugs. Examples of arylguanidines or thiazoles include abafungin (Abasol®).

Polyenes are naturally occurring compounds with a very broad antifungal spectrum. Polyenes act by binding to sterols in the fungal cell membrane, thereby interfering with membrane integrity and causing leakage of essential metabolites. Most polyenes are used topically, but intravenous amphotericin remains an important agent for the treatment of systemic fungal infections. The risk of nephrotoxicity limits the use of amphotericin B. Examples of polyenes include amphotericin B (Fungizone, Fungilin®, AmBisome®), nystatin (Nilstat®), natamycin (pimaricin), trichomycin (hachimycin).

Echinocandins are the most recently developed class of antifungals. Echinocandins are used mainly for the treatment of severe, invasive Candida infections. Echinocandins are safer than other classes of antifungals and have a broad spectrum, and synergistic effect in combination therapy. Examples of polyenes include anidulafungin (Eraxis®), caspofungin (Cancidas®), micafungin (Mycamine®).

Other potential antifungal drugs for the hydrogel system of the present invention include thiocarbamates (such as tolnaftate (Tinactin®, Aftate®, Breezee®, Ting®)); antimetabolites (such as flucytosine (Ancobon®)); benzylamines (such as butenafine (Mentax®, Lotrimin®); and griseofulvin (Gris-PEG®, Grifulvin®, Grisactin®); ciclopirox (Ciclodan®, Loprox®, Penlac®, Loprox®); selenium sulfide (Selsun®, Exsel®); tavaborole (Kerydin®); among others.

### Combination Therapy

Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the agent(s). Combination therapy may decrease the likelihood that antifungal resistance will develop. In addition to antifungal drugs, antimicrobial peptides can also be encapsulated or covalently tethered to the hydrogel backbone. Furthermore, other therapeutics such as antibiotics, endothelial growth factors, hormones, or clotting factors can be encapsulated to also aid in wound healing. In some embodiments, the therapeutic agent(s) may be directly encapsulated during the gelation process by mixing the molecule with the precursor solutions.

### Administration Methods

The compositions of the instant invention may be administered by methods well known to those skilled in the art. Such methods include local or systemic administration. In some embodiments, administration is topical. Such methods include ophthalmic administration and delivery to mucous membranes (including vaginal and rectal delivery), pulmonary (including inhalation of powders or aerosols; intratracheal, intranasal, epidermal and transdermal), oral or parenteral. Parenteral administration includes intravenous, intraarterial, subcutaneous, intraperitoneal or intramuscular injection or infusion; or intracranial (including intrathecal or intraventricular, administration).

Pharmaceutical compositions and formulations for topical administration include but are not limited to ointments, lotions, creams, transdermal patches, gels, drops, suppositories, sprays, liquids and powders. Utilization of conventional pharmaceutical carriers, oily bases, aqueous, powder, thickeners and the like may be used in the formulations. The pharmaceutical compositions may also be administered in tablets, capsules, gel capsules, and the like. Further, the responsive hydrogel material can be used to coat a wide range of medical device surfaces to prevent biofilm formation and subsequent medical device related infections. For example, but not by way of limitation, different surfaces (including but not limited to, glass, plastic, and metals) can be etched and functionalized with 10% 3(trimethoxysilyl)propyl methacrylate (TMSPMA) in acetone. This allows covalent attachment of Ac-PEG-responsive peptide-PEG-Ac conjugates to the surface of the material using the photopolymerization technique described in the Example section, thus forming thin hydrogel coatings.

Penetration enhancers may also be used in the instant pharmaceutical compositions. Such enhancers include surfactants, fatty acids, bile salts, chelating agents, and non-chelating non-surfactants. Such enhancers are generally described in U.S. Pat. No. 6,287,860.

In some embodiments, the hydrogels are delivered locally either via implantation or as an injection procedure, potentially through syringes or catheters. Anti-fungal treatment methods comprise administration of the instant compositions by any appropriate method to a patient in need of such treatment. In some embodiments, the patent is a mammal. In certain embodiments, the patient is a human.

### EXAMPLES

The following examples are given by way of illustration and are in no way intended to limit the scope of the present invention.

### Example 1 Preparation of the Biocompatible Hydrogel

### LFFK Synthesis and Characterization

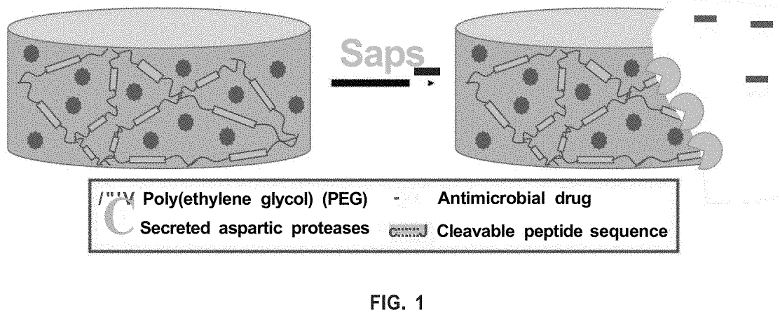

Virulent C. albicans secrete aspartic proteases (Saps) that aid in pathogen tissue invasion and proliferation. The biocompatible hydrogels of the present invention take advantage of this by degrading in the presence of these Saps, releasing the loaded therapeutic in a triggered manner (see FIG. 1). Delivering the antifungal on-demand can help in the prevention of drug resistance and reduce off-site toxicity.

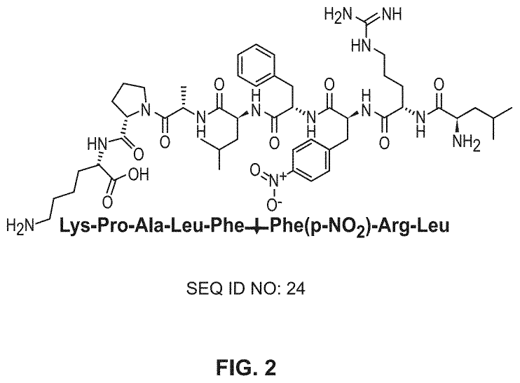

The peptide LFFK (FIG. 2), which has been shown to be readily cleaved by C. albicans Saps, was synthesized using solid-phase peptide synthesis with standard FMOC chemistry using a protocol adapted from Coin, et al.¹⁹ Briefly, a polystyrene resin, rink amide 4-methylbenzhydrylamine (MBHA), was swollen in dimethylformamide (DMF) for 30 minutes. The resin's FMOC-protected amine was deprotected using a 20% piperidine solution in DMF for 20 minutes. Following a wash with DMF, a solution of 0.4 M methylmorpholine in DMF containing HBTU as the carboxylic acid activator and the first amino acid (lysine) in the form COOH-AA-NF-FMOC, both at a 0.4 mmol concentration, was added to the resin and left to react for 30 minutes under nitrogen bubbling. The reaction was repeated for each subsequent amino acid and each amino acid was coupled twice prior to FMOC removal to increase peptide purity. Lastly, the peptide was cleaved from the resin and its side-chain protecting groups removed using a solution consisting of trifluoroacetic acid (TFA), phenol, triisopropylsilane (TIPS), and water at an 8.8/0.5/0.5/0.2 ratio, respectively. The peptide was then precipitated and washed in cold diethyl ether, dried using a rotary evaporator, dissolved in water, dialyzed, and lyophilized.

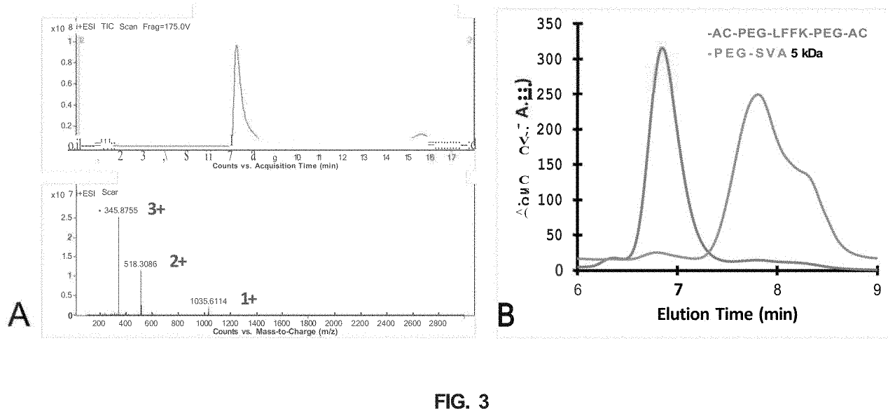

Liquid chromatography-mass spectrometry (LC-MS) and matrix assisted laser desorption ionization-time of flight (MALDI-TOF) mass spectroscopy were used to characterize peptide molecular weight and purity.²⁰ FIG. 3A depicts LC-MS results for the LFFK peptide showing the C-18 liquid chromatogram (top) and the mass spectrometry analysis (bottom). A single peak eluted at 7.5 min and the +1, +2, and +3 ionization states confirmed the expected LFFK molecular weight of 1035.56 Da. FIG. 3B depicts the size exclusion chromatography of a representative conjugate and PEG starting material. The shift to the left confirms conjugation with an average molecular weight of 11.112±0.132 Da (n=3) which includes the expected average conjugate molecular weight of 11.036 Da.

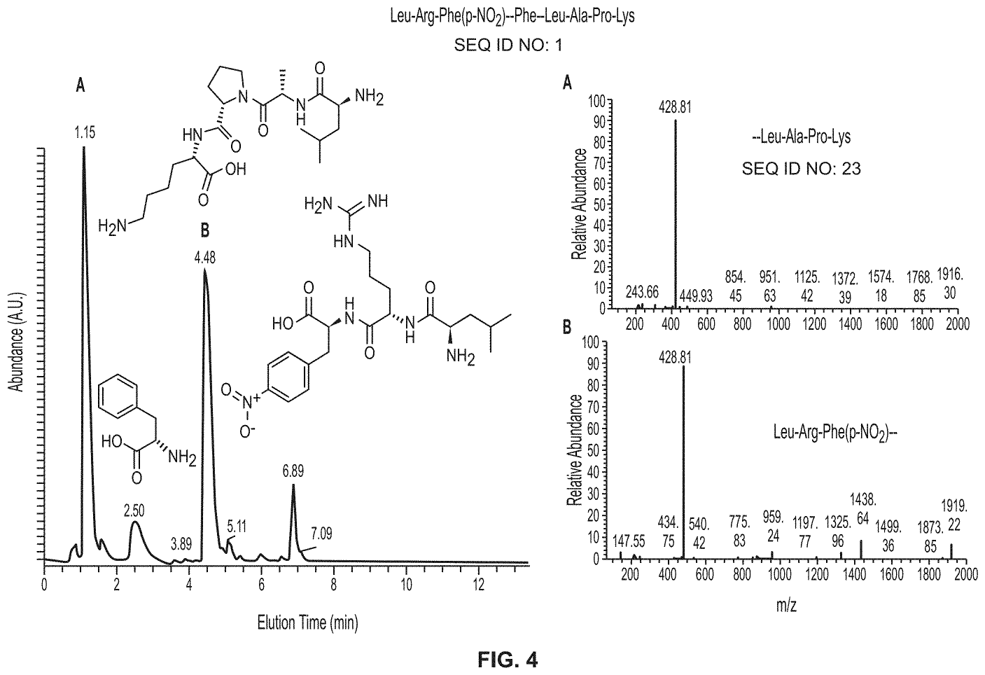

After the LFFK peptide was synthesized, its sensitivity towards aspartic proteases was evaluated in vitro. Initially, the conjugate was cultured at 37° C. with pepsin, a mammalian aspartic protease. The solution was analyzed using LC-MS to detect any residual peptide fragments. This experiment was repeated with secreted aspartic proteases extracted from C. albicans. As shown in FIG. 4, the synthesized LFFK peptide was degraded in the presence of aspartic proteases.

### PEG-LFFK-PEG Synthesis and Characterization

After the peptide was fully characterized, it was reacted with PEG (5 kDa) functionalized with succinimidyl valerate at one end, and methyl acrylate at the other end at a LFFK peptide:PEG ratio of 1 to 2. Under basic conditions, the reactive amines on the C-terminal lysine and N-terminal leucine performed a nucleophilic substitution at the PEG succinimide-activated carboxylic acid carbon, forming amide bonds. After letting the reaction proceed overnight, it was dialyzed and lyophilized.

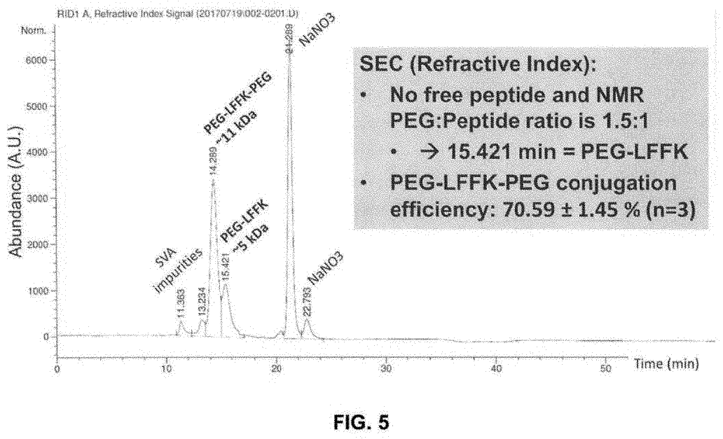

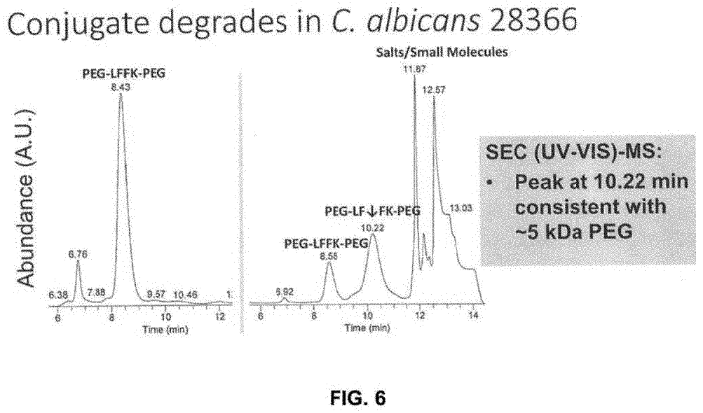

The resulting polymer was characterized using MALDI-TOF and size exclusion chromatography (SEC).²¹ The responsive LFFK peptide was successfully conjugated to PEG to form the responsive hydrogel backbone (FIG. 3B) with conjugation efficiencies of 73.73±3.21% (n=3), with no free LFFK peptide detected (FIG. 5). The degradation rate of the conjugate in the presence of Saps from C. albicans 28366 compared to free peptide was evaluated using quartz crystal microbalance with dissipation (QCM-D) to note any changes in enzyme kinetics that might result from the conjugation of PEG at lysine and leucine.^{22,23} As illustrated in FIG. 6, the analysis confirmed that the PEG-LFFK-PEG conjugate was also degraded in the presence of aspartic protease from C. albicans 28366.

### Hydrogel Fabrication

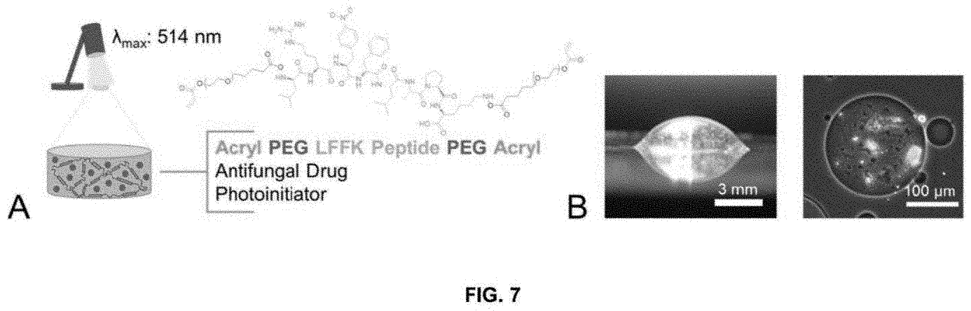

The biodegradable hydrogel system encapsulating an antifungal was fabricated via free radical photopolymerization of Acrylate-PEG-LFFK-PEG-Acrylate (PK) using eosin y and triethanolamine as co-radical initiators (see FIG. 7A). For the bulk hydrogels, the polymer backbone and model drug, anidulafungin, were dissolved in HEPES buffered saline (HBS). Eosin Y, triethanolamine (TEOA), and N-vinylpyrrolidone (NVP) were then added as the white light photoinitiator, co-initiator, and radical propagator, respectively. After thoroughly mixing the pre-polymer solution and pipetting it into a clear poly(dimethylsiloxane) (PDMS) mold, the solution was exposed to high intensity white light to trigger the photopolymerization. To form drug encapsulating microparticles, a similar method was utilized. Differences included using a dual-photoinitiator and emulsion technique.²⁴ Briefly, the pre-polymer solution was added dropwise to an oil solution containing the hydrophobic and UV light photoinitiator 2,2-dimethoxy-2-phenylacetophenone. The mixture was then emulsified by vortexing at high speeds and exposed simultaneously to white and UV light. Immediately thereafter, HBS buffer was added to the oil and the vial was centrifuged to collect the microparticles. Pluronic, dextran, and magnesium sulfate was added to tune particle size and size distribution.^{24,25} Shown in FIG. 7B is a drug loaded bulk hydrogel (left) and a drug loaded microparticle (right).

### Example 2 Assessment of the Biocompatible Hydrogel

Degradation and Release with Exposure to C. albicans

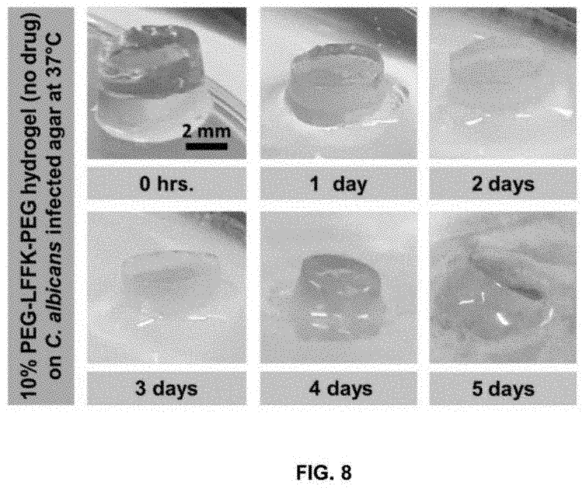

Initially, hydrogel degradation was evaluated over agar infected with C. albicans 10231 to simulate an infected wound environment. These 10% (w/v) PEG-LFFK-PEG hydrogels contained no drug and degraded in approximately 5 days (FIG. 8). The reduction in hydrogel degradation time was likely the result of the hydrogel only being exposed to C. albicans on the bottom surface of the hydrogel, which was in contact with the agar, as opposed to being submerged in a solution of Saps.

Subsequent degradation and release studies were conducted using secreted aspartic proteases extracted from C. albicans.

Sap Extraction from Candida albicans

In order to stimulate Sap production, C. albicans ATCC 10231 was inoculated in yeast carbon base supplemented with bovine serum albumin (BSA) as the sole source of nitrogen.²⁶ Protease extraction protocol was adapted from Germaine, et al.²⁷ Briefly, after 3-5 days in culture at 28° C. and shaking at 120 RPM, the solution was transferred to conical tubes and centrifuged at 4° C. for 30 minutes at 5,000 RPM. The solution supernatant was decanted into a large bottle on ice to preserve enzyme stability. Then, 61.5% (w/v) ammonium sulfate was slowly added to the culture supernatant under gentle stirring. After adding the ammonium sulfate, the solution was allowed to sit for an hour to ensure maximal enzyme precipitation. The solution was then once again transferred to conical tubes and centrifuged at 4° C. for 20 minutes at 15,000 RPM to pellet enzymes. The supernatant was decanted and the pellet dissolved in 1 mM potassium phosphate and dialyzed against the same buffer at 4° C. overnight. Finally, the solution was frozen and lyophilized to dryness. Extracted Sap molecular weight was analyzed using SEC.

In order to evaluate extracted Sap proteolytic activity, the method of Schreiber et al. was used.²⁸ A known concentration of Saps was dissolved in 25 mM sodium citrate buffer (SCB) (pH 3.2). This solution was mixed at a 1:4 ratio with a 1% (w/v) BSA solution in 25 mM SCB and incubated at 37° C. for 3 hours. The reaction was stopped by adding twice the solution volume of 5% (w/v) trichloroacetic acid and chilled on ice. Any undigested albumin precipitated. The vials were centrifuged at 2,000 RPM for 20 minutes at 4° C. and the absorbance of the soluble peptides was measured using a plate reader set to 280 nm. One proteolytic activity unit (PU) was defined as the proteolytic activity needed to generate an OD280 of 0.01 for a pathlength of 1 cm per hour.²⁹

Degradation and Release with Exposure to C. albicans Saps

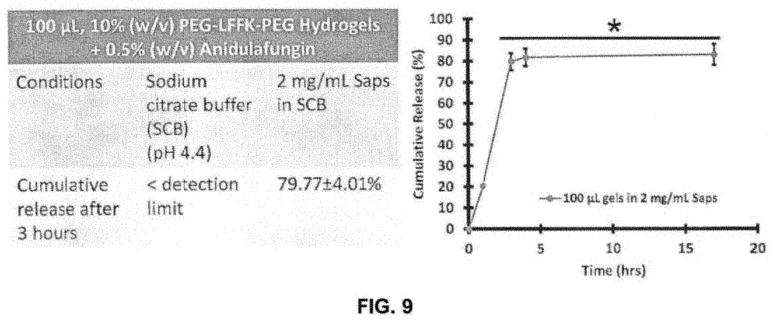

Degradation and release studies were conducted by placing the hydrogels in sodium citrate buffer (SCB) pH 4.4 with a 2 mg/mL concentration of C. albicans secreted aspartic proteases (Saps). At given time points, the solution was removed and replaced with fresh SCB with 2 mg/mL Saps. The concentration of released anidulafungin was measured using a fluorescence plate reader (λex: 250 nm, λem: 420 nm). As shown in FIG. 9, 10% PK hydrogels loaded with 0.5% (w/v) anidulafungin were degraded and released the loaded antifungal drug within three hours when exposed to a 2 mg/mL concentration of C. albicans secreted aspartic proteases. No degradation was observed when the hydrogels were incubated in sodium citrate buffer (SCB) containing no Saps at 37° C., and only 0.43±0.01% of the total loaded drug was released in 24 hours.

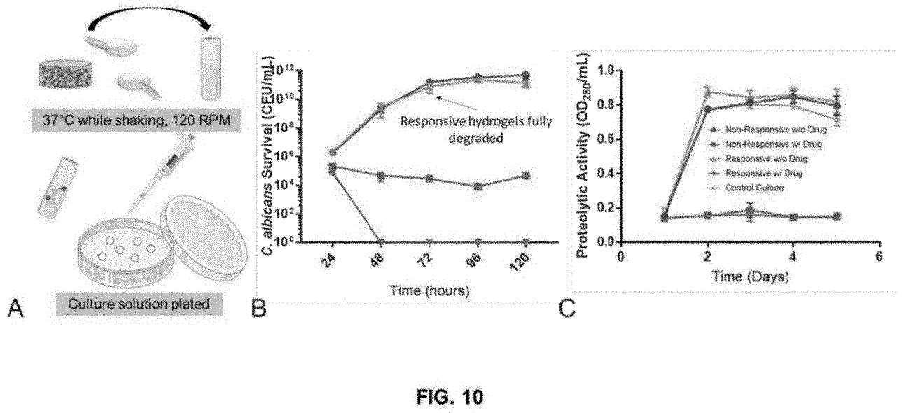

PK hydrogels loaded with a different antifungal drug, 0.05% amphotericin B encapsulated in liposomes (AmBisome), behaved similarly to those loaded with anidulafungin, releasing 92.31±0.68% and 81.76±4.15% of their total loaded drug after 4 hours in Saps at 37° C., respectively. Ten percent PK hydrogels loaded with AmBisome were exposed directly to C. albicans 10231. Hydrogels were added to a culture of 10⁷ CFU/mL C. albicans in Sap-inducing media and cultured while agitating at 37° C. (FIG. 10A). After 48 hours, responsive hydrogels loaded with AmBisome were able to fully eradicate Candida. Non-responsive hydrogels loaded with AmBisome did not achieve the fungal burden of 10¹¹ CFU/mL seen with blank hydrogels, they instead inhibited Candida growth keeping it at approximately 10⁵ CFU/mL for 5 days (FIG. 10B). Maximal proteolytic activity was achieved by the control cultures within 48 hours. Proteolytic activity was minimal for hydrogels loaded with AmBisome (FIG. 10C).

### Hydrogel Specificity

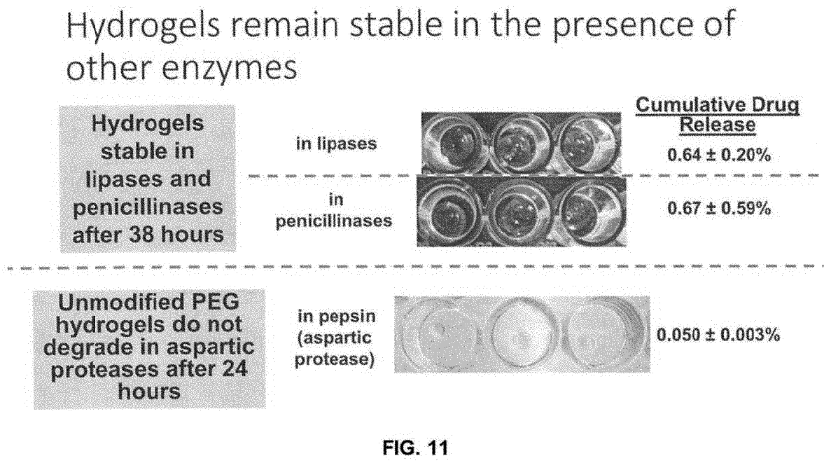

Sap-hydrogel specificity was confirmed by testing other enzymes that might be present at an infection site. As shown in the upper panel of FIG. 11, PK hydrogels proved to be stable in penicillinases and lipases, releasing only 0.67±0.59% and 0.64±0.20% of the total loaded drug, respectively, after 24 hours. Non-responsive PEG hydrogels (i.e., lacking LFFK in the backbone) remained intact in aspartic proteases for at least 24 hours, releasing just 0.05±0.003% of the total loaded drug (FIG. 11, lower panel). Other enzymes tested included human and bacterial proteases, as well as by adding Sap inhibitors such as pepstatin.

### Hydrogel Modifications

The mechanical properties of the hydrogel, namely Young's modulus, the shear modulus, and viscosity, were assessed for the different hydrogel formulations in an effort to better understand how structure relates to drug release properties. Compression testing was used to determine hydrogel elastic modulus. The hydrogels, of cylindrical shape with a height of 3.5 mm and a radius of 3 mm, were compressed between two flat steel plates to 50% strain at a rate of 0.1 mm/s. As seen with other PEG hydrogel systems, an increase in stiffness going from 5% (w/v) PEG-LFFK-PEG to 20% (w/v) PEG-LFFK-PEG was observed. Conversely, a decrease in hydrogel stiffness was observed when using larger molecular weight PEG chains to form the PK conjugates. These observations were supported by data in Durst C A, et al.³⁰

Ultimately, an increase in the elastic modulus correlated to an increase in cross-linking density and a decrease in pore size, drug diffusivity, and enzyme penetration.³¹ Hydrogel mesh size was calculated using the Peppas-Merrill model (adapted from the Flory-Rehner model) in conjunction with the Canal-Peppas model.¹⁸

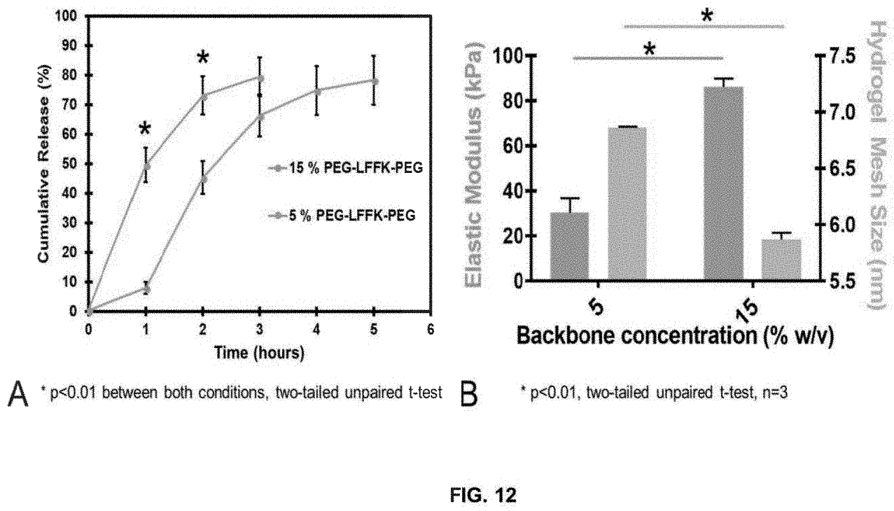

Hydrogels with different concentrations of PK were compared to assess the effects of different hydrogel mesh size on the rate of degradation and drug release. Hydrogels fabricated with 5% and 15% (w/v) PK with 0.05% (w/v) anidulafungin released 49.60±5.85% and 7.93±2.03% of the total loaded drug, respectively, after 1 hour in Saps extracted from C. albicans ATCC strain 10231 at 37° C. (FIG. 12A). The concentration of Saps mimicked proteolytic activity of clinical C. albicans isolates. The difference in degradation rates can be explained by looking at hydrogel mechanical properties and mesh size; 15% PK hydrogels were significantly stiffer and had smaller pore sizes than 5% PK hydrogels, presumably delaying enzyme penetration into the interior of the hydrogel structure, slowing degradation (FIG. 12B).

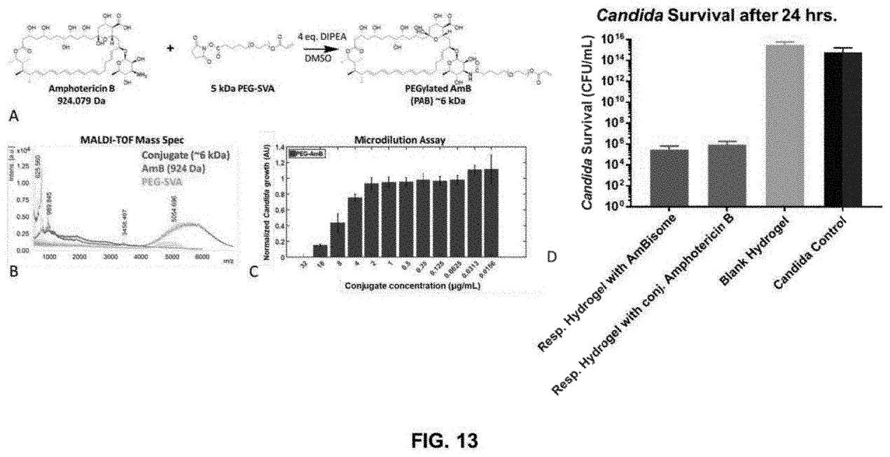

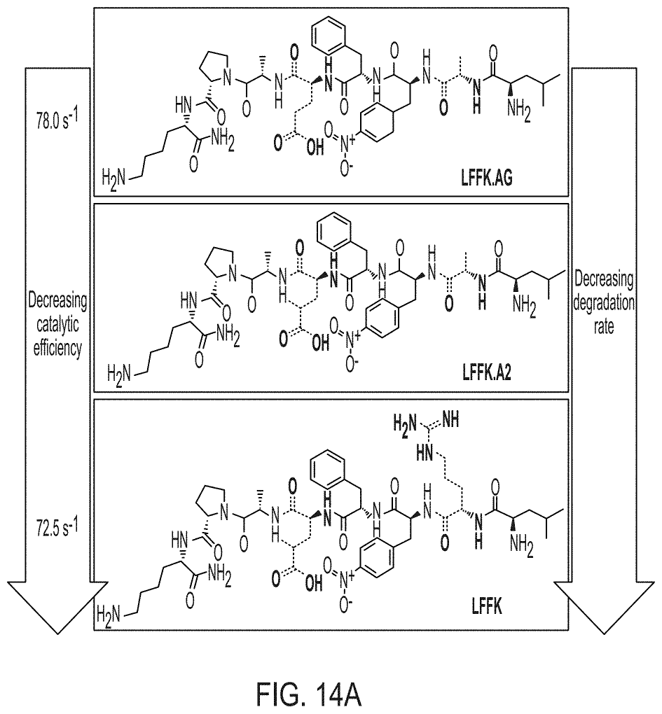

Responsive hydrogels were also by the covalent conjugation of the antifungal drug amphotericin B with acrylated PEG (FIG. 13A). The drug-PEG conjugation was confirmed with matrix-assisted laser desorption/ionization-time of flight mass spectroscopy chromatogram noting the shift in molecular weight (FIG. 13B). A micro-dilution assay demonstrated the efficacy of the drug-PEG conjugate in inhibiting Candida growth at 32 μg/mL (FIG. 13C). Responsive hydrogels with covalently linked Amphotericin B were as efficacious as responsive hydrogels encapsulating AmBisome (liposomal Amphotericin B) in reducing Candida burden after 24 hours compared to blank hydrogels and a Candida control (FIG. 13D).

Hydrogel sensitivity towards Saps was also studied by incubating the hydrogels at different Sap concentrations. The prospect of achieving an on-off response with the hydrogels was investigated by exposing them to cyclically high and low Sap concentrations. Finally, examination of photopolymerization times, ratio of responsive to non-responsive PEG, polymer concentration and molecular weight effect on hydrogel degradation and drug release were tested.

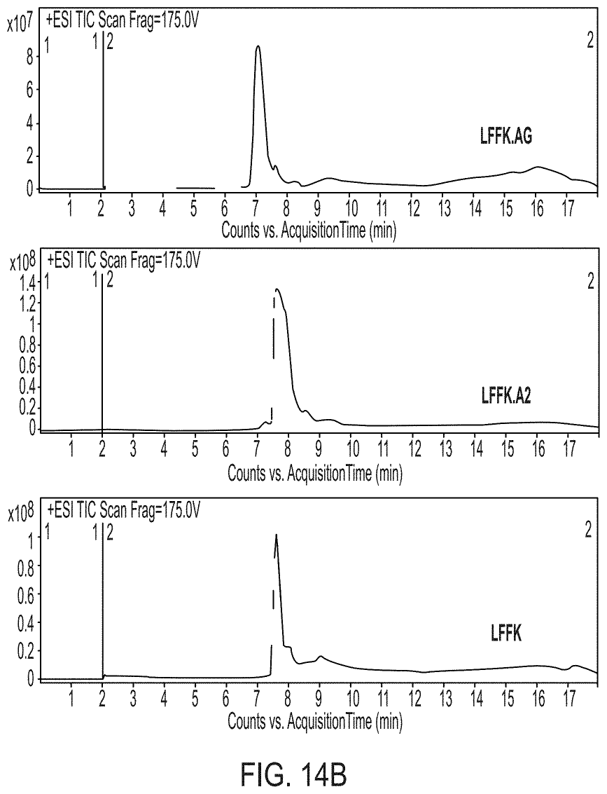

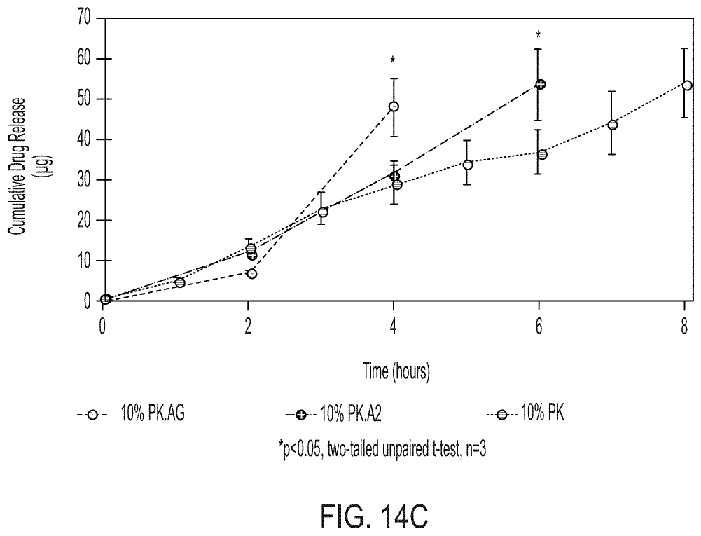

Finally, in order to further optimized the hydrogel degradation rates, LFFK was slightly altered to obtain peptides with lowered peptide-Sap catalytic efficiencies. Two variant peptides, LAF(p-NO₂)FGAPK (SEQ ID NO: 21) (LFFK.AG) and LAF(p-NO₂)FLAPK (SEQ ID NO: 22) (LFFK.A2) along with LFFK were synthesized using standard solid phase synthesis with FMOC chemistry. In order of decreasing catalytic efficiencies, these peptides were: LFFK.AG, LFFK.A2, and LFFK (FIG. 14A). The peptides were then analyzed using LC-MS (FIG. 14B). As expected, hydrogels fabricated with LFFK.AG degraded the fastest in Saps, followed by LFFK.A2, and LFFK (FIG. 14C).

Other useful peptides for Sap-triggered hydrogels include: LRFFLAPK (SEQ ID NO:2), LRF(p-NO₂)↓FKAPK (SEQ ID NO: 3), LRFFKAPK (SEQ ID NO: 4), LRF(p-NO₂)↓FAAPK (SEQ ID NO: 5), LRFFAAPK (SEQ ID NO: 6), LRF(p-NO₂)↓FDAPK (SEQ ID NO: 7), LRFFDAPK (SEQ ID NO: 8), LRF(p-NO₂)↓FRAPK (SEQ ID NO: 9), LRFFRAPK (SEQ ID NO: 10), LRF(p-NO₂)↓FKDPK (SEQ ID NO: 11), LRFFKDPK (SEQ ID NO: 12), LRF(p-NO₂)↓FKRPK (SEQ ID NO: 13), LRFFKRPK (SEQ ID NO: 14), LRF(p-NO₂)↓FEIPK (SEQ ID NO: 15), LRFFEIPK (SEQ ID NO: 16), LAF(p-NO₂)↓FEAPK (SEQ ID NO: 17), and LAFFEAPK (SEQ ID NO: 18), VFILWRTE (SEQ ID NO: 19) and TFSYnRWPK (SEQ ID NO: 20).

All references disclosed herein are incorporated by reference in their entirety.

### REFERENCES

1. Eggimann, P., J. Garbino, and D. Pittet. Epidemiology of Candida species infections in critically ill non-immunosuppressed patients. Lancet Infect. Dis. 2003; 3: 685-702.
2. U.S. Centers for Disease Control, Antibiotic Resistance Threats in the United States, 2013; p. 46.
3. Jarvis, W. R. and W. J. Martone. Predominant pathogens in hospital infections. J. Antimicrob. Chemother. 1992; 29: 19-24.
4. Richards, M. J., J. R. Edwards, D. H. Culver, R. P. Gaynes, and National Nosocomial Infections Surveillance System. Nosocomial infections in coronary care units in the United States. Am. J. Cardiol 1998; 82: 789-793.
5. Horn, D. L., D. Neofytos, E. J. Anaissie, et al. Epidemiology and outcomes of candidemia in 2019 patients: data from the prospective antifungal therapy alliance registry. Clin. Infect. Dis. 2009; 48:1695-1703.
6. Hsu, F. C., P. C. Lin, C. Y. Chi, et al. Prognostic factors for patients with culture-positive Candida infection undergoing abdominal surgery. J. Microbiol. Immunol. Infect. 2009; 42:378-384.
7. Zaidi, K. U., A. Mani, V. Thawani, and A. Mehra. Total protein profile and drug resistance in Candida albicans isolated from clinical samples. Mol. Biol. Int. 2016; 2016:4982131.
8. Mishra, N. N., T. Prasad, N. Sharma, A. Payasi, R. Prasad, D. K. Gupta, and R. Singh. Pathogenicity and drug resistance in Candida Albicans and other yeast species—A review. Acta Microbiol. Immunol. Hung. 2007; 54(3):201-235.
9. Magill S S, Edwards J R, Bamberg W, et al. Multistate point-prevalence survey of health care-associated infections. The New England journal of medicine 2014; 370:1198-1208.
10. Morgan J., M. I. Meltzer, B. D. Plikaytis, et al. Excess mortality, hospital stay, and cost due to candidemia: a case-control study using data from population-based candidemia surveillance. Infection control and hospital epidemiology 2005; 26:540-547.
11. Vallabhaneni S., A. Cleveland, M. Farley, et al. Epidemiology and Risk Factors for Echinocandin Nonsusceptible Candida glabrata Bloodstream Infections: Data From a Large Multisite Population-Based Candidemia Surveillance Program, 2008-2014. Open Forum Infect Diseases 2015; 2(4):ofv163.
12. Lockhart S. R., N. Iqbal, A. A. Cleveland, et al. Species identification and antifungal susceptibility testing of Candida bloodstream isolates from population-based surveillance studies in two U.S. cities from 2008 to 2011. Journal of clinical microbiology 2012; 50:3435-3442.
13. Alexander B. D., M. D. Johnson, C. D. Pfeiffer, et al. Increasing echinocandin resistance in Candida glabrata: clinical failure correlates with presence of FKS mutations and elevated minimum inhibitory concentrations. Clinical infectious diseases 2013; 56:1724-1732.
14. Baddley J. W., M. Patel, S. M. Bhavnani, et al. Association of fluconazole pharmacodynamics with mortality in patients with candidemia. Antimicrobial Agents and Chemotherapy 2008; 52:3022-3028.
15. Satoh K., K. Makimura, Y. Hasumi, et al. Candida auris sp. nov., a novel ascomycetous yeast isolated from the external ear canal of an inpatient in a Japanese hospital. Microbiol Immunol 2009; 53:41-44.
16. Lockhart S. R., K. A. Etienne, S. Vallabhaneni, et al. Simultaneous Emergence of Multidrug-Resistant Candida auris on 3 Continents Confirmed by Whole-Genome Sequencing and Epidemiological Analyses. Clinical infectious diseases: an official publication of the Infectious Diseases Society of America 2017; 64:134-140.
17. Khetan S., J. S. Katz, J. A. Burdick, Sequential crosslinking to control cellular spreading in 3-dimensional hydrogels. Soft Matter, 2009; 5:1601-1606.
18. Bahney, C., T. J. Lujan, C. W. Hsu, et al., Visible light photoinitiation of mesenchymal stem cell-laden bioresponsive hydrogels. European Cells & Materials, 2011; 22:43-55.
19. Coin, I., M. Beyermann, and M. Bienert, Solid-phase peptide synthesis: from standard procedures to the synthesis of difficult sequences. Nature Protocols, 2007; 2(12):3247-3256.
20. Rappsilber, J., Y. Ishihama, and M. Mann, Stop and go extraction tips for matrix-assisted laser desorption/ionization, nanoelectrospray, and LC/MS sample pretreatment in proteomics. Analytical Chemistry, 2003; 75(3):663-670.
21. Kendrick, B. S., B. A. Kerwin, B. S. Chang, et al. Online size-exclusion high-performance liquid chromatography light scattering and differential refractometry methods to determine degree of polymer conjugation to proteins and protein-protein or protein-ligand association states. Analytical Biochemistry, 2001; 299(2):136-146.
22. Stair, J. L., M. Watkinson, and S. Krause, Sensor materials for the detection of proteases. Biosensors and Bioelectronics, 2009; 24(7):2113-2118.
23. Koutsopoulos, S., L. D. Unsworth, Y. Nagai, et al., Controlled release of functional proteins through designer self-assembling peptide nanofiber hydrogel scaffold. Proceedings of the National Academy of Sciences, 2009; 106(12):4623-4628.
24. Franco, C., J. Price, and J. West, Development and optimization of a dual-photoinitiator, emulsion-based technique for rapid generation of cell-laden hydrogel microspheres. Acta Biomaterialia, 2011; 7(9):3267-3276.
25. Franssen, O. and W. E. Hennink, A novel preparation method for polymeric microparticles without the use of organic solvents. International Journal of Pharmaceutics, 1998; 168(1):1-7.
26. Hube, B., M. Monod, D. A. Schofield, et al., Expression of seven members of the gene family encoding secretory aspartyl proteinases in Candida albicans. Molecular Microbiology, 1994; 14(1):87-99.
27. Germaine, G. R., L. M. Tellefson, and G. L. Johnson, Proteolytic activity of Candida albicans: action on human salivary proteins. Infection and Immunity, 1978; 22(3):861-866.
28. Schreiber, B., C. A. Lyman, J. Gurevich, et al., Proteolytic activity of Candida albicans and other yeasts. Diagnostic Microbiology and Infectious Disease, 1985; 3(1):1-5.
29. Braga, A. A., P. B. de Morais, and V. R. Linardi, Screening of yeasts from Brazilian Amazon rain forest for extracellular proteinases production. Systematic and Applied Microbiology, 1998; 21(3):353-359.
30. Durst C A, M. P. Cuchiara, E. G. Mansfield, et al., Flexural characterization of cell encapsulated PEGDA hydrogels with applications for tissue engineered heart valves. Acta Biomaterialia, 2011; 7(6):2467-2476.
31. Bae, K. H., L.-S. Wang and M. Kurisawa, Injectable biodegradable hydrogels: progress and challenges. Journal of Materials Chemistry B, 2013; 1(40):5371-5388.

The foregoing written specification is considered to be sufficient to enable one skilled in the art to practice the present aspects and embodiments. The present aspects and embodiments are not to be limited in scope by examples provided, since the examples are intended as a single illustration of one aspect and other functionally equivalent embodiments are within the scope of the disclosure. Various modifications in addition to those shown and described herein will become apparent to those skilled in the art from the foregoing description and fall within the scope of the appended claims. The advantages and objects described herein are not necessarily encompassed by each embodiment. Those skilled in the art will recognize, or be able to ascertain using no more than routine experimentation, many equivalents to the specific embodiments described herein. Such equivalents are intended to be encompassed by the following claims.

What is claimed is:

1. A biocompatible hydrogel comprising:
(a) a plurality of cross-linkers covalently connecting the biocompatible hydrogel backbone components;
wherein the cross-linker comprises a peptide sequence that is selectively cleaved by aspartic proteases secreted by virulent, pathogenic Candida; and
wherein the biocompatible hydrogel backbone components are selected from the group consisting of: polyethylene glycol, polyester, poly(ethylene oxide), polyurethane, gellan gum, and pectin; and
(b) an antifungal therapeutic agent encapsulated within the hydrogel;
wherein the antifungal therapeutic agent is encapsulated in a liposomal nanoparticle, and
wherein the liposomal nanoparticle remains encapsulated within the hydrogel until the peptide sequence is selectively cleaved by aspartic protease, such that the cleavage by aspartic protease results in the controlled release of the antifungal therapeutic agent.

2. The biocompatible hydrogel according to claim 1, where the peptide sequence has 5-100 amino acid residues.

3. The biocompatible hydrogel according to claim 1, where the peptide sequence is selected from the group consisting of LRF(p-NO2)↓FLAPK (SEQ ID NO: 1), LRFFLAPK (SEQ ID NO: 2), LRF(p-NO2)↓FKAPK (SEQ ID NO: 3), LRFFKAPK (SEQ ID NO: 4), LRF(p-NO2)↓FAAPK, (SEQ ID NO: 5) LRFFAAPK (SEQ ID NO: 6), LRF(p-NO2)↓FDAPK (SEQ ID NO: 7), LRFFDAPK (SEQ ID NO: 8), LRF(p-NO2)↓FRAPK (SEQ ID NO: 9), LRFFRAPK (SEQ ID NO: 10), LRF(p-NO2)↓FKDPK (SEQ ID NO: 11), LRFFKDPK (SEQ ID NO: 12), LRF(p-NO2)↓FKRPK (SEQ ID NO: 13), LRFFKRPK (SEQ ID NO: 14), LRF(p-NO2)↓FEIPK (SEQ ID NO: 15), LRFFEIPK (SEQ ID NO: 16), LAF(p-NO2)↓FEAPK (SEQ ID NO: 17), LAFFEAPK (SEQ ID NO: 18), VFILWRTE (SEQ ID NO: 19), and/or TFSYnRWPK (SEQ ID NO: 20),
wherein the ↓ symbol is the cleavage point for the aspartic protease, and
wherein n refers to the amino acid norleucine.

4. The biocompatible hydrogel according to claim 1, where the peptide sequence is LRF(p-NO₂)↓FLAPK [SEQ ID NO: 1], wherein the ↓ symbol is the cleavage point for the aspartic protease.

5. The biocompatible hydrogel according to claim 1, where the antifungal therapeutic agent is selected from the group consisting of: an allylamine, an imidazole, a triazole, an arylguanidine, a polyene, an echinocandin, a thiocarbamate, an antimetabolite, a benzylamine, griseofulvin, ciclopirox, selenium sulfide, and tavaborole.


SEQUENCE LISTING (23 sequences)

SEQ ID NO: 1 - protein, 8 aa, Artificial Sequence
  Description of Artificial Sequence Synthetic peptide
  MOD_RES (3)..(3): Phe(p-NO2)
LRFFLAPK

SEQ ID NO: 2 - protein, 8 aa, Artificial Sequence
  Description of Artificial Sequence Synthetic peptide
LRFFLAPK

SEQ ID NO: 3 - protein, 8 aa, Artificial Sequence
  Description of Artificial Sequence Synthetic peptide
  MOD_RES (3)..(3): Phe(p-NO2)
LRFFKAPK

SEQ ID NO: 4 - protein, 8 aa, Artificial Sequence
  Description of Artificial Sequence Synthetic peptide
LRFFKAPK

SEQ ID NO: 5 - protein, 8 aa, Artificial Sequence
  Description of Artificial Sequence Synthetic peptide
  MOD_RES (3)..(3): Phe(p-NO2)
LRFFAAPK

SEQ ID NO: 6 - protein, 8 aa, Artificial Sequence
  Description of Artificial Sequence Synthetic peptide
LRFFAAPK

SEQ ID NO: 7 - protein, 8 aa, Artificial Sequence
  Description of Artificial Sequence Synthetic peptide
  MOD_RES (3)..(3): Phe(p-NO2)
LRFFDAPK

SEQ ID NO: 8 - protein, 8 aa, Artificial Sequence
  Description of Artificial Sequence Synthetic peptide
LRFFDAPK

SEQ ID NO: 9 - protein, 8 aa, Artificial Sequence
  Description of Artificial Sequence Synthetic peptide
  MOD_RES (3)..(3): Phe(p-NO2)
LRFFRAPK

SEQ ID NO: 10 - protein, 8 aa, Artificial Sequence
  Description of Artificial Sequence Synthetic peptide
LRFFRAPK

SEQ ID NO: 11 - protein, 8 aa, Artificial Sequence
  Description of Artificial Sequence Synthetic peptide
  MOD_RES (3)..(3): Phe(p-NO2)
LRFFKDPK

SEQ ID NO: 12 - protein, 8 aa, Artificial Sequence
  Description of Artificial Sequence Synthetic peptide
LRFFKDPK

SEQ ID NO: 13 - protein, 8 aa, Artificial Sequence
  Description of Artificial Sequence Synthetic peptide
  MOD_RES (3)..(3): Phe(p-NO2)
LRFFKRPK

SEQ ID NO: 14 - protein, 8 aa, Artificial Sequence
  Description of Artificial Sequence Synthetic peptide
LRFFKRPK

SEQ ID NO: 15 - protein, 8 aa, Artificial Sequence
  Description of Artificial Sequence Synthetic peptide
  MOD_RES (3)..(3): Phe(p-NO2)
LRFFEIPK

SEQ ID NO: 16 - protein, 8 aa, Artificial Sequence
  Description of Artificial Sequence Synthetic peptide
LRFFEIPK

SEQ ID NO: 17 - protein, 8 aa, Artificial Sequence
  Description of Artificial Sequence Synthetic peptide
  MOD_RES (3)..(3): Phe(p-NO2)
LAFFEAPK

SEQ ID NO: 18 - protein, 8 aa, Artificial Sequence
  Description of Artificial Sequence Synthetic peptide
LAFFEAPK

SEQ ID NO: 19 - protein, 8 aa, Artificial Sequence
  Description of Artificial Sequence Synthetic peptide
VFILWRTE

SEQ ID NO: 20 - protein, 9 aa, Artificial Sequence
  Description of Artificial Sequence Synthetic peptide
  MOD_RES (5)..(5): Norleucine
TFSYLRWPK

SEQ ID NO: 21 - protein, 8 aa, Artificial Sequence
  Description of Artificial Sequence Synthetic peptide
  MOD_RES (3)..(3): Phe(p-NO2)
LAFFGAPK

SEQ ID NO: 22 - protein, 8 aa, Artificial Sequence
  Description of Artificial Sequence Synthetic peptide
  MOD_RES (3)..(3): Phe(p-NO2)
LAFFLAPK

SEQ ID NO: 23 - protein, 4 aa, Artificial Sequence
  Description of Artificial Sequence Synthetic peptide
LAPK